(19) United States
(12) Patent Application Publication
(10) Pub. No.: US 2009/0157036 A1
(43) Pub. Date: Jun. 18, 2009

(54) Absorbent Article With Composite Sheet Comprising Elastic Material

(76) Inventors: Ekaterina Anatolyevna Ponomarenko, Bad Soden (DE); Mattias Schmidt, Idstein (DE); Tina Brown, Cincinnati, OH (US); Alexander Eberhard Unger, Kelkheim (DE); Alexander Berk, Frankfurt/M (DE); Darrell Ian Brown, Mason, OH (US); Blanca Arizti, Frankfurt/M (DE)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/333,668

(22) Filed: Dec. 12, 2008

Related U.S. Application Data
(60) Provisional application No. 61/013,510, filed on Dec. 13, 2007.

(51) Int. Cl. A61F 13/15; B30B 15/34; F28F 5/00; B29C 65/00

(52) U.S. Cl. 604/385.24; 604/385.01; 100/92; 492/46; 156/183

(57) ABSTRACT

A process is claimed for making a composite sheet useful for an absorbent article, the composite sheet comprising a wrinkled, patterned elasticized region that comprises a patterned first sheet and an elastic material, the first sheet being patterned with troughs, that are typically compacter, i.e. of higher density, prior to attachment to the elastic material. The troughs are attached to the elastic material. The resulting composite sheet has a uniform wrinkle pattern. Also claimed are specific absorbent articles.

### CROSS REFERENCE TO RELATED APPLICATIONS

This application claims the benefit of U.S. Provisional Application No. 61/013,510 filed on Dec. 13, 2007, which is herein incorporated by reference in its entirety.

### FIELD OF THE INVENTION

The present disclosure is directed to specific wrinkles, patterned elasticized composite sheets and a process to make specific such composite sheets, the sheets having an improved wrinkle profile; the present disclosure also relates to absorbent articles comprising such composite sheet materials.

### BACKGROUND OF THE INVENTION

It is well known that absorbent articles may comprise leg cuffs or barrier cuffs, typically made of a barrier material and typically being elasticized for better fit and better sealing. Other components of an absorbent article may also be elasticized, such as for example the waistband. These elasticized components are typically in contact with the wearer's skin. Another example of an elasticized component of an absorbent article is a so-called anal and/or vaginal cuff, also referred to as elasticated or elasticized topsheet with one or more openings. This can serve to isolate fecal material, blood and/or urine away from the skin, e.g. under the cuff. This isolation is beneficial, because such bodily exudates, and in particular fecal material, are often difficult to remove from the skin of the user, in particular from sensitive skin such as that of young babies and the skin around the genitals. Moreover, it is well known that such bodily exudates on the skin can cause irritation and redness of the skin and sometimes even dermatitis of the skin. Thus, isolating the fecal material, blood, or urine away from the skin can reduce such skin problems. Typically, barrier materials are used for such cuffs.

Materials that provide a good barrier are often not very comfortable in use. These barrier materials are sometimes difficult to elasticize. It may be desirable that cuffs (but also any other elasticized component of absorbent articles) have softer elastic regions for sensitive skin. Ideally, the wrinkles caused by the elastics leave no or minimal pressure marks. It has been found that the wrinkles should therefore be very uniform. However, it has been found to be difficult to produce elasticized articles with uniform elastic profile and uniform wrinkles, in particular when produced at high speed, or when the elastics are applied in a curvilinear pattern, or when the elastics are applied under high strain, or when the material that is to be elasticized is thick or stiff, such as may be the case with high barrier materials.

The inventors found that with prior art processes, whereby the elastic material is merely attached in stretch state to a nonwoven with adhesives, the attachment of the elastic material in stretched state may result in irregular attachment areas and subsequently irregular wrinkles.

The present disclosure provides an improved process to provide comfortable elasticized composite sheet materials and an improved elasticized composite sheet material useful for absorbent articles. In the improved process, the wrinkle formation is controlled, by submitting a first sheet to a specific patterning step to form troughs, which are then simultaneously attached by application of gentle pressure to a stretched elastic material. The resulting patterned and wrinkled composite sheet material has an improved wrinkle pattern that may be softer or more comfortable for the user.

### SUMMARY OF THE INVENTION

The present disclosure relates to a process for making a composite sheet useful for an absorbent article, the composite sheet comprising a wrinkled, patterned elasticized region that comprises a patterned first sheet and an elastic material, the composite sheet being obtainable by:
a) obtaining a first sheet with a Y-direction;
b) obtaining an elastic material that is at least partially stretched, having at least an average longitudinal direction of stretch in the Y-direction (average-direction stretch);
c) i) submitting the first sheet or part thereof to a patterning, pressure-applying step to obtain a patterned first sheet comprising troughs, (e.g. substantially along the y-direction), and then positioning the at least partially stretched elastic material adjacent the patterned first sheet to obtain a combined material; or
   ii) positioning the at least partially stretched elastic material adjacent the first sheet to obtain a combined material and simultaneously or subsequently submitting the combined material, or part thereof, to a patterning, pressure-applying step to obtain a patterned combined material, comprising a patterned first sheet comprising troughs (e.g. substantially along the Y-direction);
d) simultaneously or subsequent to step c) attaching the thus formed troughs, or part thereof, of the patterned first sheet to the elastic material, to thus obtain a stretched composite sheet comprising a patterned elasticized region;
e) relaxing the composite sheet of step d) to obtain a composite sheet comprising a wrinkled, patterned elasticized region, comprising wrinkles with peaks and valleys, the valleys being formed by both the elastic material and the troughs of the first sheet.

While the process disclosed herein may be used to make any composite sheet comprising an elastic material and a first sheet, or comprising even additional sheet materials, the process is in particular useful to make composite sheets that have a barrier first sheet, as described herein; a thick or stiff first sheet, having the bending rigidity as described herein; curved or angled elastics, e.g. curvilinear elastics; elasticized regions, whereby adhesion of the elastic material and first sheet is achieved by application of pressure and optionally heat (and for example without adhesive present); and/or elastic material applied under high strain.

The process may for example be used to make composite sheets that comprise a first sheet that is a nonwoven sheet, comprising one or more nonwoven layers that is/are (each) a laminate of one or more spunbond and one or more meltblown webs; and/or a first sheet, having the hydrostatic head values and/or bending rigidity values, described herein.

In one embodiment, the process comprises step c ii) and the first sheet and elastic material are positioned adjacent one another and simultaneously or subsequently the first sheet is patterned; the attachment of the troughs may then be achieved simultaneously with the patterning step.

In one embodiment, the surface of the first sheet that is not facing the elastic material is pressurized by, and possibly directly contacted by, a first patterning tool's surface having raised portions and the opposite surface of the first sheet, which faces the elastic material, is pressurized, and may be indirectly contacted, by a second, non-mating surface of a tool, for example an even surface; thus, the first tool's surface may be the surface of a patterned roll with raised portions and the second tool's surface being the surface of an anvil roll with non-mating raised portions, or may be without raised portions, e.g. having an even surface.

The troughs of the first sheet (i.e. the portions of the first sheet that form the troughs) is typically more compact, having a higher density, than the portions of the first sheet not forming the troughs, (e.g. the portions of the first sheet between neighboring troughs, including the crests and optionally portions of the first sheet that are not patterned and/or that are not pressurized by the patterning tool).

It has furthermore been found that the provision of a pattern of troughs in the first sheet and selectively attaching these troughs to the elastic material allows very effective attachment of the first sheet to a minimum amount of elastic material (while this is in stretched state), thus resulting in a minimum of elastic material that remains in stretched state after relaxation of the composite sheet. Thus, optimum strength of the attachment of the elastic material to the first sheet can be achieved, while at the same time optimum elasticity can be maintained.

In one embodiment an absorbent article may include a composite sheet that includes a wrinkled and patterned elasticized region, the region containing a first sheet and an elastic material, the elasticized region having a residual strain of less than 30%, less than 20%, or between 2% and 20%, and in one embodiment herein when a second sheet is present, having a peel force of at least 1.4 N, or at least 2.0 N, or at least 2.4N, or at least 3.0N.

The process may be such that the pressure between the first tool's surface and the second tool's (i.e. second) surface, or between the first surface of the first tool and the first sheet, is from 20,000 to 80,000 psi.

The tool may comprise studs or teeth placed in rows substantially along the X-direction of the tool and placed in columns along substantially the Y-direction of the tool, and therefore, the composite sheet material and elasticized region thereof may comprises a multitude of troughs along the Y-direction (one or more column of troughs) and along the x-direction (rows of troughs) of the composite sheet.

The process and resulting composite sheet may be such that the troughs of the first sheet have a higher density than the portions of the first sheet not forming the troughs, e.g. the portions not contacted by the tool.

The elastic material may, for example, be an elastic band with an average width of 3 mm to 25 mm and the patterned first sheet may have a pattern with a width that is from 70% to 300% of the width of the elastic material.

Adhesive may be used to attach the elastic material to the first sheet. In one embodiment, the adhesive is applied as thin filaments, e.g. having an average diameter of less than 200 microns.

The elastic material may be a slow-recovery elastomer, and then adhesive may even be omitted.

In one embodiment, the composite sheet has an elastic profile of: 1.5 Lt by a first load force of less than 1.1N, 3.0 Lt by a first load force of less than 2.1N and 4.5 Lt by a first load force of less than 3.0N and a second unload force at 4.5 Lt of more than 0.9N, a second unload force at 3.0 Lt of more than 0.5N and a second unload force at 1.5 Lt of more than 0.1N.

### BRIEF DESCRIPTION OF THE FIGURES

FIG. 1 shows a perspective view of an absorbent article herein, including a composite sheet having patterned, wrinkles elasticized regions.

FIG. 1a shows a perspective view of a portion A of the article of FIG. 1.

FIG. 2a shows a cross-sectional view of a fully stretched elasticized region of a composite sheet herein, having a first sheet that is patterned.

FIG. 2b shows the elasticized region of FIG. 2a in partially stretched state.

FIG. 2c shows the elasticized region of FIG. 2a in contracted, relaxed state.

FIG. 3a shows a cross-sectional view of an alternative elasticized region of a composite sheet herein, in fully stretched state, having a first sheet and a second sheet that are patterned.

FIG. 3b shows the elasticized region of FIG. 3a in partially stretched state.

FIG. 3c shows the elasticized region of FIG. 3a in contracted, relaxed state.

FIG. 4 shows a perspective view of a first tool and second tool that may be used herein to form the patterned composite sheet.

FIG. 5 shows a perspective view of an alternative first tool and second tool that may be used herein to form the patterned composite sheet.

### DETAILED DESCRIPTION

As used herein, the following terms have the following meanings:

“Absorbent article” means any article that can absorb body fluids and is suitable to be placed in close proximity to or against the skin of a user, e.g. the genitals and/or anus of the user (including in particular feminine hygiene articles and adult, baby, or infant diapers or pads, including baby, infant, toddler diapers with fasteners, and so-called training or pull-up pants and adult incontinence articles).

As used herein “front region” and “back region” refer to the two regions, which are in use, respectively, closest to the front of the wearer and the back of the wearer.

As used herein, the term “void space” is a cavity in the article present in at least the relaxed state, which serves to accept and contain bodily exudates such as fecal material, having, for example, a volume of at least 3 or even 5 cm³ in relaxed state.

When used herein, “longitudinal direction” or “longitudinal dimension” is a direction or dimension, respectively, running “substantially parallel” to the maximum linear direction or dimension of the sheet or article. This is indicated as direction Y, unless stated otherwise.

The “lateral direction”, or “lateral dimension”, or “transverse direction” or “transverse dimension” is the direction or dimension, respectively, perpendicular to the longitudinal direction or dimension, respectively, and in the same plane as the majority of the article or (composite) sheet and the longitudinal axis. This is indicated as direction X.

“Substantially perpendicular” and “substantially parallel” include directions within 30°, and may be within 20 or 15°, from the exact perpendicular or parallel direction, unless stated or specified otherwise.

“Direction of stretch” when used herein is considered the average direction of stretch.

As used herein, “along” means at least partially (substantially) parallel to and adjacent to.

“Adjacent” includes in close proximity with and in contact with.

As used herein “relaxed” or “relaxed state” or “contracted” or “contracted state” means the state that no forces are applied to the article, to the composite sheet, to the elasticized region, or elastic material, or to the waistband/cuff herein (other than naturally occurring forces such as gravity), e.g. when it is laid on a horizontal surface.

### Composite Sheet (10) and Process for Making the Composite Sheet (10) and First Tool Used Thereby

The composite sheet (10) herein comprises at least a first sheet (13) and an elastic material (15). Where both the elastic material (15) and the first sheet (13) are present (e.g. one overlaying the other) an elasticized region (12 a and/or 12 b) is formed. The first sheet (13) may be wider, in the transverse direction, than the elastic material (15). The first sheet (13) may also be attached to two or more elastic materials (15), and there may thus be two or more elasticized regions (12 a, b) in a composite sheet (10).

The composite sheet (10) and the first sheet (13) or part thereof (e.g. all or part of the area of the first sheet (13) forming the elasticized region) is patterned, comprising a multitude of troughs (16). This may be done by use of a patterning tool (30) that pressurizes, and may contact, the first sheet (13) with raised portions (32) extending from the surface (31) of the tool to form the troughs (16) of the first sheet. The first sheet (13), once present in the composite sheet (10), thus comprises typically a multitude of troughs (16) and a multitude of crests (17).

The portions of the first sheet (13) forming the troughs (16) may be compacted by this patterning step, being thus compacted trough (18) (or compacted attachment areas 18); thus, the portions of the first sheet (13) forming the troughs (16) may have a higher density than the portions of the first sheet (13) not forming the trough, e.g. the crests (17), and/or the portions of the first sheet (13) that may not be patterned.

In one embodiment herein, the first sheet (13) or part thereof is patterned in step c ii) and comprises troughs (16), but the elastic material (15) is not patterned and/or the elastic material (15) does not comprise troughs (16). In another embodiment, the elastic material (15) may be patterned in step c ii) but this is less than the patterning of the first sheet, i.e. such that the troughs (16) of the elastic material (15) as obtained by the patterning step are smaller in height than the troughs (16) of the first sheet, typically at least 50% less. This may for example be applicable when the elastic material (15) comprises a thermoplastic component and the patterning step involves the application of heat.

In relaxed state, the patterned elasticized region is a wrinkled elasticized region (12), due to the contraction forces of the elastic material (15) that cause the patterned first sheet (13) and patterned elasticized region (12) to wrinkle. Due to the patterning step, the valleys (22) of the wrinkles will coincide with the troughs (16), as also further described herein (and the crests (17) of the first sheet (13) with the peaks (21) of the wrinkles).

The elastic material (15) is applied to the first sheet (13) in stretched or partially stretched state, for example, the elastic material (15) is at least stretched to 150% or at least 200% of its fully contracted length, when applied to the first sheet; it may be stretched to at least 250% of its original, contracted length, or at least 300% or at least 330%, but it may be less than 600%.

In one embodiment herein, the elastic material is placed in a diverging manner, with typically a straight portion substantially in the Machine Direction (MD), that is the Y-direction, of the article or first sheet thereof, and one or more diverging portions that are under an angle with the MD or Y-direction and with the straight portion. The straight portion may be at least present in the centre ⅓ of the length or part thereof of the article or sheet (crotch portion), and/or the straight portion may be at least 20% or at least 30% of the total length of the elastic material. In such an embodiment, the description herein of the troughs, crests, peaks and valleys apply to this straight portion at least, and optionally also to the diverging portion(s). Furthermore, the description herein of the tool and the raised portions thereof applies in such an embodiment at least in so far it is placed in close proximity with the straight portion.

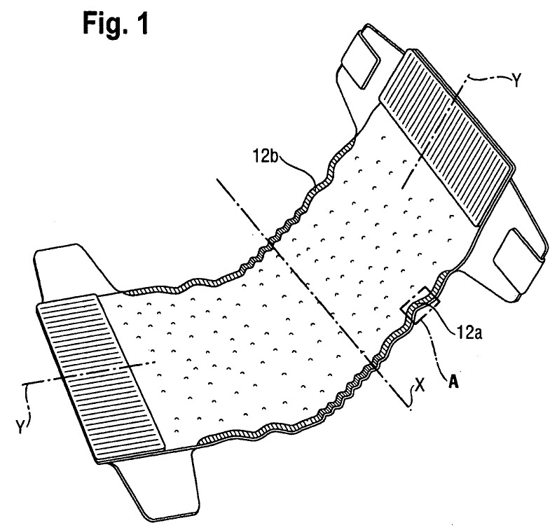

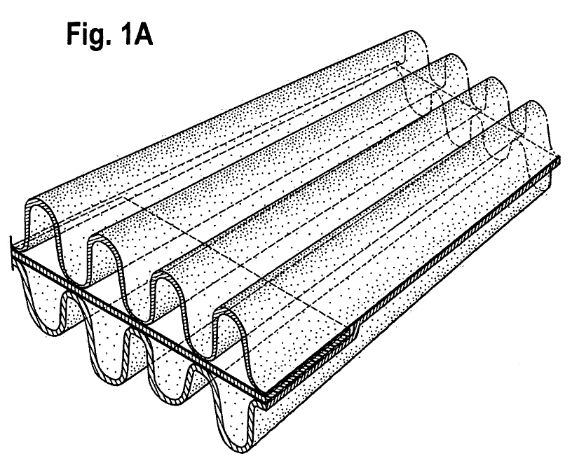

In one embodiment, the elastic material (15) is positioned at least partially substantially parallel to the longitudinal Y-direction of the first sheet or article, forming a straight portion, as described above. At least 30% or at least 50% of its length may be positioned substantially parallel to the Y-direction. The elastic material may have an average direction of stretch or elasticity substantially parallel to the longitudinal direction. In one embodiment, the elastic material (15) is applied along a curvilinear pattern, for example, such that the elastic material (15) crosses a, or each, transverse axis of the composite sheet (10) only once, and the longitudinal axis thereof at least twice. This is for example shown in FIG. 1.

The portion of the first sheet (13) that is submitted to the patterning step, or the first sheet (13) as a whole, may not be elastic prior to the patterning step and the step of applying the elastic material (15).

The patterned first sheet (13) may comprise the pattern of troughs (16) over part or all of its length (in longitudinal direction Y) and/or over all or part of its width (in transverse direction X) of the sheet (13) or of the elasticized region (12) thereof. Thus, the first sheet (13) may also comprise the pattern outside the elasticized region (12).

The first sheet and/or elasticized region (12 a, b) may comprise at least one, or only one, trough in transverse direction X and, for example, at least 15 troughs, or at least 50 troughs, or at least 100 troughs in longitudinal direction of the region, sheet and/or article.

At least 10% of the length of the first sheet (13) that forms the elasticized region (12) may comprise the pattern of troughs (16) at least 30% or even at least 40% or at least 60% or at least 75% or at least 90% or even about 100% of its length.

At least 30% of the width of the part of the first sheet (13) that forms the elasticized region (12) may comprise the pattern of troughs (16), or, for example, at least 50% or at least 70% or at least 80% or at least 90% or even about 100%. Furthermore, the width of the part of the first sheet (13) that comprises the troughs (16) may be more than the width of the elasticized region (12) (and more than the width of the elastic material (15)), for example, from 100% to 500% of the average width of the elastic elasticized region (12), or from 100% to 250% or from 100% to 150%. The troughs may be present over substantially the whole width of the first sheet (13).

The number of troughs (16) per cm along the elasticized region (12) substantially in the Y-direction may vary; in one embodiment, the elasticized region (12) and/or the first sheet (13) has in contracted state an average of from 5 to 25 troughs per cm, or from 5 to 20 troughs per cm, or from 7 to 15 troughs per cm.

The patterning step may be done by applying, indirectly or directly, a patterning surface of a first tool (30) to the surface of the first sheet (13) that does not face the elastic material (15) (but that faces typically in use the user's skin). This tool surface (31) may be a continuous surface, and the tool is for example a patterned roll (30). The first tool's surface (31) comprises raised portions (32).

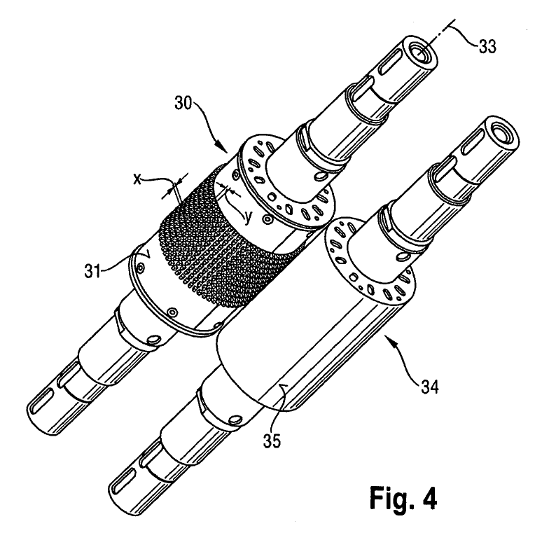

Each raised portion has a width dimension X, substantially parallel to the width of the tool surface, (and typically substantially parallel to the axis (33) of the tool, when the tool is a roll), as shown for example in FIG. 4. Each raised portion has also a length dimension Y, substantially parallel to the length of the tool surface, (and typically substantially perpendicular to the axis (33) of the tool, when the tool is a roll), as shown for example in FIG. 4.

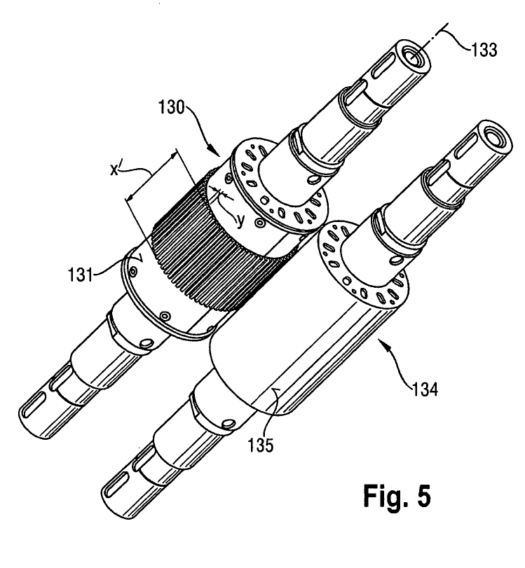

The raised portions (32) may have any shape, for example they may be in the form of studs, or teeth, as for example shown in FIG. 4, and/or ridges, as for example shown in FIG. 5. The surface may have rows of studs and/or teeth substantially along the X-direction, for example at least 2, or at least 3, or at least 4 of such raised portions per row; whereby the surface may also comprise the raised portions in columns in the Y-direction (substantially perpendicular to the X-direction), and typically over the length dimension Y of the surface. It may be that the pattern of the raised portions is such that the columns of raised portions in Y-direction are not aligned, so that the pattern is staggered or alternating.

The opposite surface of the first sheet (13), which faces the elastic material (15), is pressurized and may be indirectly contacted by a surface (35) of a second tool (34), to apply a counter pressure to the first tool's surface (31). This second tool's surface (35) may be even or non-mating with the first tool's surface (31). The second surface (35) may also be a continuous second surface such as a surface of a second roll (34). The second tool's surface (35) may comprise raised portions that contact the raised portions of the first tool (30) in a non-mating manner. In one embodiment, the second tool's surface (35) does not comprise raised portions and may have an even surface. The second tool (34) may be an anvil roll (34). The elastic material (15), or the second sheet (14) described hereinafter may be directly contacted by a second tool (34), such as the anvil roll (34).

One process according to the present disclosure comprises the step c ii) and not step c i), and thus the features disclosed herein apply to the step c ii).

The patterning step may apply a pressure that is large enough to ensure patterning of the first sheet (13) and contacting of the thus formed troughs (16) with the elastic material (15) and possibly aiding attachment thereof to the elastic material (15). The applied pressure may be minimized, to avoid attachment of the crests (17) of the first sheet (13) to the elastic material (15). Suitable pressures may depend on the first sheet's properties, including chemistry of the first sheet, bending rigidity, thickness, and on elastic material (15) properties, including chemistry, thickness; the pressure may also be adjusted depending on whether other attachment means are used, such as heat, or adhesive.

The average pressure applied by the raised portions (32) onto the first sheet (13), or onto the second tool surface (34), may, for example, be from 10,000 to 100,000 psi, or from 20,000 to 80,000 psi, or for example from 30,000 to 60,000 psi (obtainable by calculation).

The average distance between the highest point of a raised portions (in x-y plane) of the first tool (30) and highest point of the surface of the second tool (34) may be from 0.01 mm to 1.0 mm, or from 0.025 mm to 0.6 mm, or to 0.5 mm, or to 0.3 mm, or to 0.25 mm.

As mentioned above, the patterning surface of the first tool (30) may comprise raised portions of any shape. The raised portions may, for example, be studs, teeth, or ridges. Such raised portions have a Z-dimension, e.g. the height of the raised portions, the Z-dimension being perpendicular to the X- and Y-dimensions of the first tool, e.g. perpendicular to the axis of the tool and out of plane of the tool, when the tool is a roll.

In one embodiment the raised portions include a first flat (distal) surface (in x-y plane) that in use contacts the sheet material first. This surface may have a Y-dimension (substantially parallel to the MD of the sheet and/or substantially perpendicular to the X-direction of the tool, e.g. the axis of the tool of, for example, 0.05 mm to 0.5 mm, or from 0.1 mm or 0.2 mm to 0.4 mm. The X-dimension of such first flat surfaces may be, for example, from 0.05 to 2.0 mm, or to 1.5 mm; or from 0.1 mm or 0.2 mm to 0.5 mm or to 0.4 mm.

If the raised portion is a ridge, it may be present over substantially (e.g. 90% or more) of the width (X-direction) of the tool, and its dimension in X-direction may thus be substantially the same as the width of the tool.

Within a column, the Y-direction distance between the highest point of a raised portion, or alternately the center point of a flat surface of a raised portion, as described above, and the highest point of a neighboring raised portion or the centre point of a flat surface of a neighboring raised portion, in the Y-direction may be, in one embodiment, from 1.0 mm to 3.0 mm, up to 2.0 mm, or for example from 1.3 mm to 2.0 mm. This is hereinafter referred to as the periodicity of the raised portions in Y-direction.

Within a row, the X-direction distance between the highest point of a raised portion, or alternately the center point of a flat surface of a raised portion, as described above, and the highest point of a neighboring raised portion or the center point of a surface of a neighboring raised portion in the direction X, may be, in one embodiment from 0.2 mm to 2.0 mm, up to 1.5 mm, or for example from 0.5 mm to 1.5 mm, or from 0.7 mm to 1.3 mm.

In another embodiment (in particular when the X-direction of the tool is placed substantially perpendicular to the average direction of stretch of the elastic material, and/or substantially perpendicular to the Y-direction or MD of the first sheet), the height of a raised portion, as measured from the lowest point of a valley between two neighboring raised portions in the X-direction to the highest point or center point of the raised portion (hereinafter referred to as X-direction-defined height), is less than the Y-direction defined height of the same raised portion, as measured from the foot of a valley between two neighboring raised portions in the Y-direction, to the highest point of the raised portion (herein referred to as Y-direction height).

The average Z-direction height of the valleys between raised portions in the Y-direction, or that all Z-direction heights of the valleys between raised portions in the Y-direction, may be more than the average Z-direction heights or than all Z-direction heights of the raised portions in the X-direction. It may for example be that the ratio of the Y-direction defined height to the X-direction defined height is at least 3:2, or at least 2:1, or at least 5:2, or at least 3:1, or at least 4:1, or at least 5:1, or for example up to 10:1, or up to 8:1.

The X-direction-defined height may, for example, be (absolute or on average) from 0.1 mm to 1.0 mm, or for example 0.2 mm to 0.6 mm, or for example, to 0.4 mm, or to 0.3 mm. The Y-direction defined height may, for example, be (on average or absolute) from 0.4 mm to 3 mm, or from 0.6 mm, or from 0.8 mm or from 1.0 mm, to 2.5 mm, or to 2.0 mm.

Thus, in some embodiments, the composite sheet, or the first sheet, or the wrinkled, patterned elasticized region thereof comprises a pattern of troughs in both the Y-direction and X-direction, whereby the trough(s) have an Y-direction height that is more than the X-direction height of the trough (the heights being defined as for the raised portions valleys above). It may, for example, be that the ratio of the Y-direction height to the X-direction height is for example at least 3:2, or at least 2:1 or at least 5:2, or at least 3:1, or at least 4:1, or at least 5:1, or for example up to 10:1, or up to 8:1.

One or more, or all valleys between neighboring raised portions may define a flat surface area (e.g. the lowest surface area of the valley), in substantially the X-Y direction.

In another embodiment, as described above, the tool comprises studs or teeth placed in rows substantially along the X-direction of the tool, and placed in columns along substantially the Y-direction of the tool, and thus, the first sheet, or elasticized regions or combined material of the absorbent article has one or more troughs along the Y-direction and along the X-direction.

In one embodiment, the composite sheet or elasticized region thereof comprises two or three or more elastic strings or strands or bands, aligned along side, each extending at least partially in substantially the Y-direction. Such a tool may comprise a multitude of teeth or studs in substantially the X-direction and Y-direction of the tool, and the tool may be placed in contact or close proximity with the first sheet and in close proximity to the elastic strands such that the valleys between studs or teeth of the tool in the X-direction correspond with the area between the elastic strands or strings in the X-direction, so that thus troughs are formed along the X-direction between the elastic strings or strands.

A composite sheet may, therefore, have a first sheet and an elasticized region with troughs in X and Y direction, and wrinkles in Y-direction, and, for example, an elasticized region with a multitude of elastic bands, extending at least partially in substantially the Y-direction and providing an elastic force in substantially the Y-direction, whereby the composite sheet comprises one or more troughs between the elastic strands, strings, or bands, and may be a single trough between two neighboring strands, strings or bands.

In one embodiment, the average caliper of the first sheet (13) is less than 50% or less than 40% or less than 25% of the average distance between the raised portions in the Y-direction, as described above.

The patterning tool may be directly or indirectly placed onto the first sheet. This may be done such that the width dimension (dimension in X-direction) of the tool is within 45°, or within 30° or within 20° or within 10° of the average transverse direction X (width) of the elasticized region (12) and/or elastic material (15), (the width and transverse direction being perpendicular to the direction of stretch).

In one embodiment, the tool (33) comprises raised portions, being ridges or rows of teeth or studs, that are each placed within 30°, or within 20° or within 10° of the line parallel to the axis of the tool (33); or in one embodiment, between 5° and 20° or 5° and 15°.

The pattern applied by the patterning tool may thus have such a corresponding angle with the elastic material: it may be that for at least 50% of the troughs, or for all of the troughs, a line parallel to the width of the troughs has an angle with the line parallel to the direction of stretch of the elastic material at the trough or parallel to the MD direction of the first sheet (Y-direction), of from 5°, or from 10°, to 40°, or to 30°, or to 25°, or to 20°.

The patterning step may also serve to adhere the elastic material to the first sheet (13). Optionally heat bonding, ultrasonic bonding and/or of adhesive may also be used. The present process allows attachment and wrinkle formation that is independent of the bonding method used, e.g. independent of the adhesive pattern used. In some embodiments, the attachment of the first sheet (13) and elastic material (15) is controlled by the patterning step and pattern thereof, and not by the adhesive pattern (as is typically the case in prior art processes for applying elastic material (15) to nonwovens).

For certain embodiments, the amount of adhesive used can be significantly reduced without reducing the strength of the bonding of the elastic material to the first sheet: for example less than 20 g/m² (per surface area of the first sheet (13) or of the elastic material), or less than 10 g/m² or less than 8 g/m² of adhesive may be used. At least 1 g/m² of adhesive may be used.

The adhesive (even when used in amount greater than described above, e.g. more than 20 g/m², as may be desirable in certain embodiments herein) may be applied as very thin filaments or fibers, e.g. the resulting elasticized region, or composite sheet may comprise adhesive filaments of an average or absolute diameter of less than 200 microns, or between 50 and 150 microns, as can be visualized in the elasticized region (12) of the composite sheet (10) by use of microscopy. Adhesives that may be satisfactorily used herein include adhesives manufactured by H. B. Fuller Company of St. Paul, Minn. and marketed as HL-1620 and HL-1358-XZP and adhesives from National Starch. The adhesive may be applied by known techniques, including spraying or melt-blowing. The adhesive may be applied in a pattern, such as a spiral or double spiral or omega pattern, or it may in one embodiment be applied randomly. However, it may be applied in a uniform amount per surface area, e.g. per cm². In one embodiment, the adhesive is applied as randomly oriented adhesive fibers in a uniform amount, e.g. as mentioned above. In another embodiment, the adhesive may be applied in a pattern of re-occurring shapes, such as a (double) spiral or (double) omega pattern. It may be that the adhesive is applied in a continuous manner, e.g. as a continuous pattern of shapes, whereby the shapes are attached to one another.

When more than one elastic string or strand is present, or when one or more elastic band is present (per elastic region), such as one or more elastic bands of an average width of 3 mm to 25 mm, the adhesive may be applied in at least two, or at least 3 parallel areas which each extend in the direction of stretch of the elastic material(s), such as, for example, at least 2, or at least 3 continuous patterns of re-occurring shapes. For example, at least 2, or at least 3, or at least 4 parallel omega or spiral adhesive patterns may be applied along the average direction of stretch of the elastic material or the Y-direction of the sheet or article, either to the elastic materials or to the sheet.

In one embodiment, the adhesive may be applied in a pattern of shapes with a certain periodicity of re-occurring patterns, which may be defined by the distance from a selected point of a shape to the same selected point on a neighboring shape, such as the distance between the highest point of an omega shape, to the highest point on a neighboring omega shape, along the Y-direction; this distance may for example be from 0.05 mm to 2.5 mm, or from 0.05 mm to 2.0 mm or to 1.7 mm. It may for example be useful to have from 5 to 14 omega patterns per cm, or for example from 5 to 12, or from 6 to 12 or to 10 (as can be measured on a the first sheet that is at its full length and width).

It may be that the ratio of the periodicity of the raised portions of the tool and/or of the periodicity of troughs of the first sheet, to the periodicity of the adhesive pattern is from 0.7, or from 0.8, or from 0.9 to 2.0, or to 1.5, or to 1.3, or it may even be about 1.0 (in substantially the Y-direction).

In one embodiment, use of adhesive may even be omitted, and the patterning step is used to apply pressure and optionally also heat, to achieve the partial attachment of the first sheet's troughs with the elastic material. The resulting composite sheet may thus have elasticized regions that are free of adhesive.

In one embodiment, the patterning step may apply heat to the first sheet (13) and/or to the elastic material (15), and the elastic material (15) and first sheet (13) may be attached to one another by the heat and the pressure, (and optionally by adhesive). In this embodiment it may be useful that the elastic material (15) comprises a thermoplastic component that adheres to the first sheet (13) under the process temperature of this step. The elastic material (15) may have an elastic component that is thermoplastic and adheres to the first sheet (13) under the process heat of the patterning step, and/or it may comprise an elastic component and a thermoplastic component, for example a thermoplastic coating on a elastomeric material, that adheres to the first sheet (13) under process temperature of the patterning step. Process temperatures may for example be between 30° C. and 165° C., or between 40° C. and 150° C. or 50° C. and 150° C.

Exemplary thermoplastic components are described below.

The process may also be done under cooling of the first sheet (13) and/or composite sheet (10) in step c), d) and/or e), e.g. under cooling of the patterning tool's surface, for example by cooling of the first tool's surface (31) with raised portions and/or by cooling of the second tools' surface. For example, the process may be done such that the first sheet (13) and/or the resulting composite sheet (10) is contacted by a surface that has a temperature of between −20° C. and 15° C. or between −20° C. and 10° C. or between −10° C. and 5° C. In one embodiment, the equipment is a combination of a first tool with a patterning surface with raised portions and an opposing second tool, whereby the first and/or second tool comprises a cooling system, the tools having the raised portions as described above, including the raised portion dimensions and X-direction valley dimensions and/or Y direction valley dimensions.

The composite sheet (10) comprises, in relaxed state, wrinkles, whereof the peaks (21) are formed by the first sheet (13) and the valleys (22) are formed by the first sheet's troughs (16) and elastic material (15).

In one embodiment, the composite sheet (10) may have an elasticized region (12) with a first sheet (13) with wrinkles that are uniform, e.g. a uniform wrinkle pattern. The first sheet (13) may have wrinkles that have a uniform wrinkle height and/or uniform wrinkle density, namely:

a) At a partial elongation of the composite sheet (10) of ε, which is 2/3 (i.e. 66.7%) of its fully stretched length, the first sheet (13) present in the elasticized region (12) may have wrinkles with an average wrinkle height H_{w} (as measured by the “Primos” method set out below, using PRIMOS equipment) of from 150 microns to 600 microns, or from at least 180 microns or from at least 200 microns up to 550 microns, or up to 500 microns. This height is the distance between the highest point of a peak of a wrinkle to the lowest point of the valley of the wrinkle of the first sheet, as described in the “method section”.

The average wrinkle height above may have a standard deviation (STD) of less than 100 microns, or less than 75 microns, or less than 50 microns and the RSD (being the STD/H_{w}) is less than 30% or less than 20% or less than 10%.

b) In addition, or alternatively, at the elongation ε of 2/3 (=66.7%) of the fully stretched length (as set out above, and measured with the “Primos method”) the first sheet (13) of the elasticized region (12) of the composite sheet (10) herein may have wrinkles such that the average distance between the highest points of neighboring peaks, or between center points of the highest regions of neighboring peaks (which ever is applicable), of the wrinkles is from 500 to 1500 microns, or from 750 to 1400 microns, or from 800 to 1300 microns, or from 900 to 1200 microns, wherein the standard deviation may be less than 250 microns, or less than 200 microns, or less than 100 microns.

The RSD (=STD/average) may hereby be less than 30%, or less than 20% or less than 10%.

c) In addition, or alternatively to b) above, at the elongation ε of 2/3 (=66.7%) of the fully stretched length (as set out above, and measured with the “Primos method”) the first sheet (13) of the elasticized region (12) of the composite sheet (10) herein may have wrinkles such that the average distance between the lowest points of neighboring valleys or between center points of the lowest regions of neighboring valleys (which ever is applicable), of the wrinkles is from 500 to 1500 microns, or from 750 to 1400 microns, or from 800 to 1300 microns or from 900 to 1200 microns, wherein the standard deviation may be less than 250 microns, or less than 200 microns, or less than 100 microns.

The RSD (=STD/average) may hereby be less than 30%, or less than 20% or less than 10%.

c) Alternatively, or in addition, at least 80%, or at least 90%, or even 100%, of the wrinkles of the first sheet (13) (as defined above and measured with the “Primos method”) have a wrinkle height between 650 microns and 150 microns, or between 600 microns and 200 microns, or between 500 microns and 200 microns or 250 microns, and may be less than 10%, or even less than 5%, of the wrinkles has a height of 700 microns or more.

d) Alternatively, or in addition, at the elongation ε of 2/3 (=66.7%) of the fully stretched length, the first sheet (13) of the elasticized region (12) herein may have an average wrinkle density of from 5 to 25 wrinkles per cm or from 6 to 20, from 6 to 15 per cm, or from 6 to 10 wrinkles per cm, or from 6 to 9 wrinkles per cm, as measured with the Primos method and as set out herein, and having a RSD of less than 30%, or less than 20%, or less than 10%.

In one embodiment, the first sheet (13) is folded around the elastic material (15) as a C-fold, and it thus serves also as covering sheet material, second sheet (14), for the opposite side of the elastic material (15).

Alternatively, or in addition, the opposite surface of the elastic material (15), not facing the first sheet (13) may be contacted and attached to an additional covering sheet material, i.e. second sheet (14).

In one embodiment herein, the first sheet (13) and elastic material (15) are positioned adjacent a second sheet (14) in any of the process steps b) c) or d); the elastic material may then be present between the first and second sheet. The second sheet (15) may be present during the patterning step.

This second sheet may thus be formed from the first sheet, or it may be an additional sheet, made of any material that is pliable and can form wrinkles under the elastic forces of the elastic material. It may be a nonwoven as described hereinafter as nonwovens for the first sheet (13). The second sheet (14) is typically attached to the elastic material (15) in stretched state, by any method, including those described for the first sheet. The second sheet (14) may be patterned with troughs (16) and attached to the elastic material (15) with the troughs (16), by the process described herein for the first sheet (13). This may be done in a separate process step prior to or after the patterning process step of the first sheet (13), or it may be done at the same time as the pattering step to pattern the first sheet. This may be done by use of the first tool (30) too, or it may be done with a second tool (34) having raised portions such that the top surface contacts the top surface of the raised portions (31) of the first tool. In one embodiment, the second sheet (14) is combined with the elastic material (15) and the first sheet material (13) to form a combined material that is then pressurized by the first tool (30) to form a pattern of troughs (16) in both the first and second sheet. The troughs may have uniform wrinkles. Either the second or the first sheet may be contacted by the first tool.

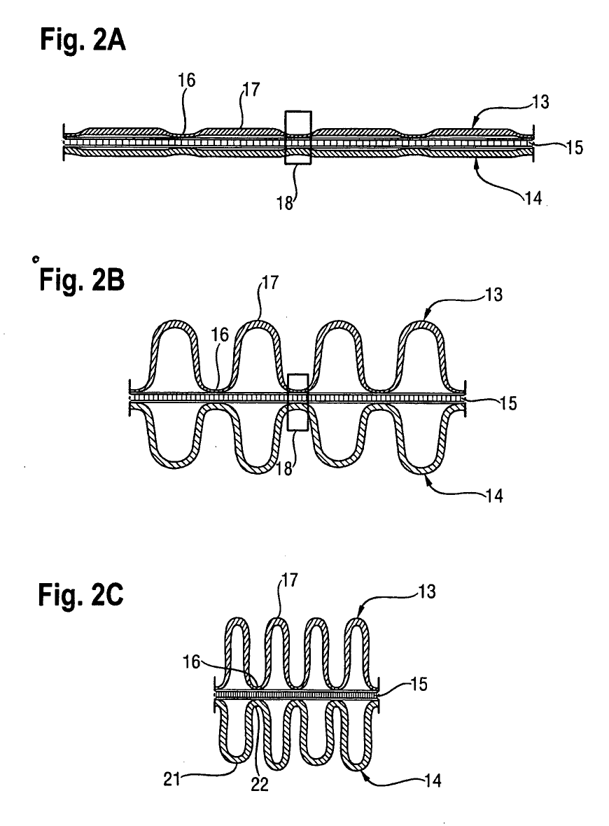

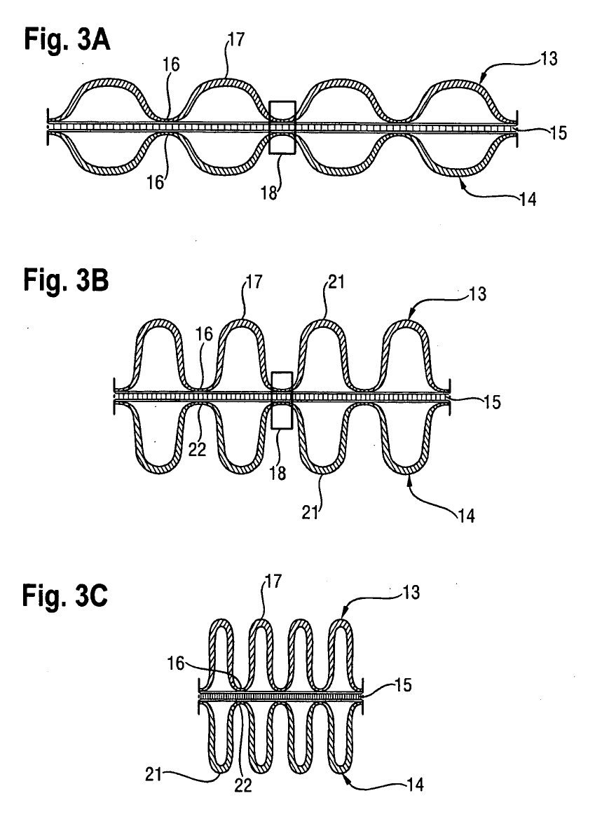

An embodiment whereby both sides of the elastic material (15) of the elasticized region (12) are contacted and adhered to, respectively a first sheet (13) and a second sheet (14) is shown in FIGS. 2A, B, C and 3A, B, C. As shown in FIGS. 3A, B, C, the second sheet (14) may also be patterned as described herein, and this may or may not be the same pattern of troughs as the pattern of troughs of the first sheet (13). It may thus also comprise troughs (16) that are compacted, having a higher density than the portions of the second sheet (14) that do not form the troughs of the second sheet (14), just as described for the first sheet (13). It may have the uniform wrinkle pattern as set out above.

Alternatively, as shown in FIGS. 2A, B, C, the second sheet (14) may not be patterned with troughs (16) and may have a non-uniform wrinkle pattern, not fulfilling the requirements as set out for the first sheet.

The composite sheet (10) that comprises a wrinkled and patterned elasticized region (12) as described having a first sheet (13) and any type of second sheet (14) may have a peel force of at least 1.5 N, or at least 2.0N or at least 2.4N, or at least 3.0N.

The elasticized region (12) may have a residual strain of less than 30%, or less than 20%, or between 1% and 30%, or between 5% and 20%, as measured by the method described below.

Elastic Material (15)

The elastic material (15) herein may be any elastic material (15) and it may be in any form or shape. The elastic material (15) may be in the form of a string, having a thickness to width ratio of 1:1 to 1:4, having for example a substantially circular cross section, or it may be in the form of a band, having a thickness to width ratio of more than 1:4. The elasticized region (12a, b) may comprise a multitude of strings or bands of elastic material. The elasticized region may have one or more straight portion(s) and one or more diverging portion(s), as described above. In one embodiment, an elasticized region comprises a central straight portion and a first end diverging portion and a second end diverging portion.

The elastic materials (15) used may be very thin, having for example a thickness or caliper (e.g. gauge) of up to about 200 microns, or even up to 150 microns, or even up to 110 microns, or up to 100 microns. The elastic material herein may have any minimum caliper, but it may be at least 20 microns, or at least 40 microns, or even at least 60 microns. The elastic material (15) may have a thickness of about 70 to 100 microns.

Suitable elastic materials may be such that they provide the following elastic profile to the composite sheet (10):

1.5 Lt by a first load force of less than 1.1N, 3.0 Lt by a first load force of less than 2.1N, and 4.5 Lt by a first load force of less than 3.0N and a second unload force at 4.5 Lt of more than 0.9N, a second unload force at 3.0 Lt of more than 0.5N, and a second unload force at 1.5 Lt of more than 0.1N.

In one embodiment herein, the elastic profile of the composite sheet (10) herein is:

1.5 Lt by a first load force of less than 0.6 N, 3.0 Lt by a first load force of less than 1.1N, and 4.5 Lt by a first load force of less than 1.5N, and a second unload force at 4.5 Lt of more than 0.9N, a second unload force at 3.0 Lt of more than 0.5N, and a second unload force at 1.5 Lt of more than 0.1N.

Lt is the length of the composite sheet (10) as set out in EP1201212-A (referred to as shortened topsheet length). EP 1 201 212A also describes the test method to measure the elastic profile.

Suitable elastic materials include cross-linked elastic polymers, including cross-linked rubbers.

A suitable elastic material (15) is for example 2L-89, available from Fulflex, (Limerick, Ireland).

The elastic material may be a so-called slow recovery elastomer, as described in co-pending application WO2006/135357A1 (PCT Application number US2005/020222). When used herein, the slow-recovery elastic is typically an elastomer which exhibits a normalized unload force at 37° C. of at least about 0.04 N/mm² as measured by the Two Cycle Hysteresis Test set out in WO2006/135357A1 (PCT Application number US2005/020222). The slow recovery elastomer exhibits at least a 20%, or at least 35%, or at least 50%, post elongation strain at 22° C. after 15 seconds of recovery, as measured by the Post Elongation Recovery Test set out in the above application.

The slow recovery elastomer may comprise about 20% to about 70%, by weight, of at least one elastomeric polymer; and the remaining portion being components such as those described below.

It may have a normalized unload force at 37° C. of at least about 0.16 N/mm² and at least a 10% post elongation strain at 22° C. after 15 seconds of recovery.

The slow recovery elastomer may exhibit a normalized unload force at 37° C. of greater than about 0.04 N/mm² and a post elongation strain of at least about 20% after 15 seconds of recovery at 22° C., as described in the co-pending application.

The slow recovery elastomer may exhibit a normalized unload force of greater than about 0.08 N/mm² at 37° C., and in one embodiment, it may exhibit a normalized unload force of greater than about 0.12 N/mm² at 37° C. In other suitable embodiments, at 22° C. the slow recovery elastomer exhibits a post elongation strain from about 75% to about 150% after 15 seconds of recovery. However, post elongation strain after 15 seconds of recovery may exceed about 170% at 22° C.

Furthermore, the slow recovery elastomers may exhibit a specified post elongation strain at 22° C. after 30 seconds, 60 seconds, or three minutes of recovery. In certain embodiments, the slow recovery elastomer may exhibit at least about a 70% post elongation strain after 30 seconds of recovery at 22° C. In some embodiments, the slow recovery elastomer may exhibit at least about a 40% post elongation strain after 60 seconds of recovery at 22° C.

Suitable elastomeric polymers comprise styrenic block copolymers, natural and synthetic rubbers, polyisoprene, neoprene, polyurethanes, silicone rubbers, hydrocarbon elastomers, ionomers, and the like. In one embodiment, the elastomeric polymer may be a block copolymer. A number of block copolymers may be used to prepare the slow recovery elastomer including multi-block, tapered block and star block copolymers. Generally, the block copolymers suitable for use in the slow recovery elastomer may exhibit both elastomeric and thermoplastic characteristics. In such block copolymers a hard block (or segment) may have a glass transition temperature (Tg) greater than about 25° C. or be crystalline or semicrystalline with a melting temperature (Tm) above about 25° C. The hard block may have a Tg greater than about 35° C. or is crystalline or semicrystalline with a Tm above about 35° C. The hard block portion is typically derived from vinyl monomers, including vinyl arenes such as styrene and alpha-methyl-styrene or combinations thereof.

Glass transition temperatures are determined by tensile dynamic mechanical analysis performed in the linear elasticized region (12) of the material at a frequency of 1 Hz using a temperature ramp method. Suitably, film samples with a uniform thickness of about 0.3 mm or less may be used with a temperature ramp rate of about 1° C./min or slower. The Tan δ peak temperature is taken as the Tg of the particular material or phase.

Crystalline melting temperatures are determined by Differential Scanning Calorimetry using a temperature ramp rate of 10° C./min. The melting endotherm peak temperature is taken as the Tm of the particular crystalline region.

The block copolymers may comprise a soft block (or segment). The soft block generally exhibits a sufficiently low glass transition temperature and/or melting temperature so as not to form glassy or crystalline regions at the use temperature of the copolymer. In one embodiment, the use temperature may be between about room temperature (about 22° C.) and about body temperature (about 32° C.). However, other use temperatures are feasible and within the scope of this invention. Such soft blocks are generally physically incompatible with the hard blocks and form separate regions, domains, or phases.

The soft block portion may be a polymer derived from conjugated aliphatic diene monomers. Typically, the soft block monomers contain fewer than about 6 carbon atoms. Suitable diene monomers include butadiene, isoprene, and the like. Particularly soft block polymers may include poly(butadiene) and poly(isoprene). Furthermore, it is envisioned that the soft block may be modified to tailor the Tg of the soft block. For example, a random copolymer of isoprene and styrene or a graft of styrene onto poly(isoprene) may be used. In such cases, lower amounts of the modifying resin may be used.

Suitable block copolymers may comprise at least one hard block (A) and at least one soft block (B). In one embodiment, the block copolymer may be an A-B-A triblock copolymer, an A-B-A-B tetrablock copolymer, or an A-B-A-B-A pentablock copolymer. Also, useful herein are triblock copolymers having endblocks A and A′, wherein A and A′ may be derived from different vinyl compounds.

Elastomeric polymers may include styrene-olefin-styrene triblock copolymers such as styrene-butadiene-styrene (S-B-S), styrene-ethylene/butylene-styrene (S-EB-S), styrene-ethylene/propylene-styrene (S-EP-S), styrene-isoprene-styrene (S-I-S), hydrogenated polystyrene-isoprene/butadiene-styrene (S-EEP-S), and mixtures thereof. The block copolymers may be employed alone or in a blend of block copolymers. Particularly, block copolymers may include styrene-butadiene-styrene (S-B-S) and styrene-isoprene-styrene (S-I-S) block copolymers. Such linear block copolymers of styrene-butadiene-styrene (S-B-S) and styrene-isoprene-styrene (S-I-S) are commercially available under the trade designation Vector from Dexco Polymers L.P., Houston, Tex., and under the trade designation Kraton from Kraton Polymers, Houston, Tex.

Modifying resins may be used; they should have a sufficiently high average molecular weight such that the glass transition temperature of the soft block is increased resulting in an increase of post elongation strain at 22° C. after 15 seconds of recovery. The slow recovery elastomer may comprise the modifying resin in amounts from about 0% to about 60% by weight. In one embodiment, the composition comprises from about 20% to about 55% or from about 35% to about 45% of the modifying resin.

Suitable modifying resins may have glass transition temperatures ranging from about 60° C. to about 180° C., or from about 70° C. to about 150° C., and or from about 90° C. to about 130° C.

Modifying resins useful herein include unhydrogenated C5 hydrocarbon resins or C9 hydrocarbon resins, partially and fully hydrogenated C5 hydrocarbon resins or C9 hydrocarbon resins; cycloaliphatic resins; terpene resins; polystyrene and styrene oligomers; poly(t-butylstyrene) or oligomers thereof; rosin and rosin derivatives; coumarone indenes; polycyclopentadiene and oligomers thereof; polymethylstyrene or oligomers thereof; phenolic resins; indene polymers, oligomers and copolymers; acrylate and methacrylate oligomers, polymers, or copolymers; derivatives thereof; and combinations thereof. The resin may be selected from the group consisting of the oligomers, polymers and/or copolymers derived from: t-butylstyrene, cyclopentadiene, iso-bornyl methacrylate, methyl methacrylate, isobutyl methacrylate, indene, coumarone, vinylcyclohexane, methylstyrene, and 3,3,5-trimethylcyclohexyl methacrylate. Modifying resins may also include alicyclic terpenes, hydrocarbon resins, cycloaliphatic resins, poly-beta-pinene, terpene phenolic resins, and combinations thereof. “C5 hydrocarbon resins” and “C9 hydrocarbon resins” are disclosed in U.S. Pat. No. 6,310,154.

The slow recovery elastomer may exhibit temperature responsiveness. In one embodiment, a temperature responsive slow recovery elastomer may exhibit a post elongation strain after 15 seconds at 32° C. that is at least 35% less than the post elongation strain after 15 seconds at 22° C.

In one embodiment, at least a 50% reduction in post elongation strain is exhibited. In another embodiment, at least a 75% reduction in post elongation strain is exhibited. A slow recovery elastomer exhibiting temperature responsiveness may further facilitate diaper application. When the absorbent article is applied at about room temperature (e.g., approximately 22° C.), the slow recovery elastomer exhibits a relatively high degree of post elongation strain for a prescribed period of time. Upon application of the diaper, the slow recovery elastomer will rise in temperature because of the close proximity of the wearer's skin. As the temperature of the slow recovery elastomer increases and nears body temperature (e.g., approximately 32° C.), the reduced post elongation strain is exhibited. Temperature responsiveness allows for application of the diaper without “snap-back” while providing for increased recovery after application.

Other components may be stabilizers, antioxidants, and bacteriostats (to avoid degradation of the slow recovery elastomer). Generally, the additive or additives may account for about 0.01% to about 60%, or to about 25%, or to about 10%, of the total weight of the slow recovery elastomer composition.

Other optional additives include thermoplastic polymers or thermoplastic polymer compositions which preferentially associate with the hard blocks or segments of the block copolymers. Without intending to be bound by theory, it is believed that these thermoplastic polymers become incorporated into the entangled three-dimensional network structure of the hard phase. This entangled network structure can provide improved tensile, elastic and stress relaxation properties of the elastomeric composition. Where the elastomeric polymer comprises a styrenic block copolymer, thermoplastic polymer additives such as polyphenylene oxide and vinylarene polymers derived from monomers including styrene, alpha-methyl styrene, para-methyl styrene, other alkyl styrene derivatives, vinyl toluene, and mixtures thereof, are useful because they are generally considered to be chemically compatible with the styrenic hard blocks of the block copolymer.

Processing aids may also be included, such as processing oils, which are well known in the art and include synthetic and natural oils, naphthenic oils, paraffinic oils, olefin oligomers and low molecular weight polymers, vegetable oils, animal oils, and derivatives of such including hydrogenated versions, such as a mineral oil. Viscosity modifiers may also be used, such as those well known in the art. For example, petroleum derived waxes can be used to reduce the viscosity of the slow recovery elastomer in thermal processing. Suitable waxes include low number-average molecular weight (e.g., 600-6000) polyethylene; petroleum waxes such as paraffin wax and microcrystalline wax; atactic polypropylene; synthetic waxes made by polymerizing carbon monoxide and hydrogen such as Fischer-Tropsch wax; and polyolefin waxes.

First Sheet (13)

The first sheet (13) may be any sheet material useful for absorbent articles, including woven sheets, nonwoven sheet, films.

In one embodiment, the first sheet (13) is not elastically extendable or stretchable, e.g. under normal process strain.

In one embodiment herein the first sheet (13) is a nonwoven sheet. The first sheet (13) may be a nonwoven sheet that is a laminate of two or more nonwoven layers and/or two or more nonwoven webs.

As used herein, a “nonwoven web” is a single web, while a “nonwoven layer” may comprise a multitude of nonwoven webs; a “nonwoven sheet” may comprise a multitude of nonwoven layers.

The first sheet (13) may be a (nonwoven) barrier sheet, such as first sheets with a hydrostatic head value (measured with the hydrostatic head test set out herein) of at least 10 mbar, or at least 15 mbar, or at least 18 mbar, or in one embodiment, at least 20 mbar, or at least 25 mbar, or at least 28 mbar, or at least 30 mbar, or in one embodiment at least 35 mbar.

In one embodiment, the hydrostatic head of the first sheet (13) is between 10 and 50 mbar.

In one embodiment, the composite sheet (10) may alternatively or in addition have the hydrostatic head values above.

The first sheet (13) or composite sheet (10) is considered to have the above hydrostatic head values if it has this value at any part of the first sheet (13) material, excluding elasticized areas or areas with edges that are attached to other materials, prior to attachment to the elastic material (15) in the process herein; and/or if it has this value at any part of the first sheet (13) after attachment to the elastic material (15), excluding at the elasticized region (12) or at areas that have edges that are attached to other materials; i.e. the measurement is done on a sample of the sheet that does not comprise elastic material (15) or edges of the first sheet (13) that are attached to another material. In one embodiment, the first sheet (13) and/or composite sheet has a surface area free of elastics or edges of at least 2.5 cm×2.5 cm.

The first sheet (13) may have a bending rigidity of 20 grams or less, or 16 grams or less, or even 14 grams or less, or 12 grams or less, as measured with the handle-o-meter test set out herein.

Alternatively, or in addition, the composite sheet (10) may have a bending rigidity of less than 35 grams, or less than 30 grams, or less than 25 grams, or less than 20 grams, or less than 18 grams, or as described above.

A first sheet (13) or composite sheet (10) herein is considered to have the above bending rigidity values if it has this value at any part of the material, excluding areas comprising elastic material, including the elasticized region (12) herein, or edges attached to other materials (these areas should not be included in the test).

The bending rigidity is the rigidity of the sheet in any direction.

In one embodiment, the first sheet (13) comprises at one surface, e.g. that is not to be attached to the elastic material (15) and that faces the patterning tool, a nonwoven web comprising fibers with an average fiber direction, and the first sheet (13) has a bending rigidity of the values specified above, in the fiber direction. Nonwoven webs that contact the patterning tool and/or that are on the surface of the first sheet (13) that is not attached to the elastic material (15) may be spunbond webs (with fibers with an average fiber direction).

The average fiber direction may typically be the longitudinal direction of the composite sheet (10) and/or the machine direction (MD) of the absorbent article.

The first sheet (13) or the composite sheet (10) has in one embodiment a low surface tension strike through value, as determined by the method described herein, of at least 30 seconds, or at least 50 seconds, or even at least 60 seconds. The strike through value may be less than 200 seconds, or less than 150 seconds, or less than 100 seconds. A first sheet (13) or composite sheet (10) is considered to have the above low surface tension strike through values if it has this value at any part of the material, excluding areas comprising elastic material (15), including the elasticized region, or edges being attached to other materials.

As mentioned above, in one embodiment the first sheet (13) is a nonwoven sheet that comprises two or more nonwoven layers that are attached to one another, but not fully (i.e. not 100%) laminated to one another. In one embodiment, the two (or more) nonwoven layers have an attachment area of 60% or less, or 40% or less, or even 20% or less, of the total area of overlap between two neighboring nonwoven layers. In one embodiment, the first sheet (13) comprises two or more nonwoven layers that are attached to one another along the side edges of the overlap area, e.g. along the edges of each or one of the nonwoven webs (periphery) and optionally the area where elastic material (15) is present, and the nonwoven layer comprises areas, e.g. of at least 0.5 cm², where both layers are present but not attached to one another. In one embodiment, the first sheet (13) is such that at least two nonwoven layers thereof are only partially attached to one another and there is at least one area of 2.5×2.5 cm that is not attached, and does not comprise elastics or edges.

In one embodiment, the first sheet (13) is a nonwoven sheet that comprises nano-fibers, which have an average diameter of 1.0 microns or less. It may be that the first sheet (13) comprises two or more nonwoven layers, whereof one or more, or each, comprise a nonwoven web that comprises such nano-fibers. The nonwoven sheet or layer or web may for example comprise at least 2 g/m² of nano-fibers, or at least 3 g/m² of nano-fibers, or at least 5 g/m² of nano-fibers. The nano-fibers may have an average diameter of 0.8 microns or less, or 0.6 microns or less. The nano-fibers may be made by known melt fibrillation methods or melt film fibrillation methods, such as described in U.S. Pat. No. 6,315,806 and U.S. Pat. No. 6,695,992. Nano-fiber webs and layers are described in co-pending application WO2005/103355.

In one embodiment, the first sheet (13) is a nonwoven sheet that has at least one nonwoven layer, comprising at least one nonwoven web of meltblown fibers, typically present at a weight level of at least 5 g/m² by weight of the nonwoven layer, or for example at least 5.7 g/m², or at least 7 g/m², but for example less than 20 g/m² or less than 15 g/m², by weight of the nonwoven layer.

The basis weight of the first sheet (13) is generally at least 5 g/m², or at least 7 g/m², or at least 10 g/m², or at least 17 g/m², or at least 22 g/m²; it may be that the basis weight is 60 g/m² or less, or 45 g/m² or less, or 40 g/m² or less, or 35 g/m² or less.

If the first sheet (13) comprises two nonwoven layers, each comprising two or more nonwoven webs, it may be that the basis weight of each of the nonwoven layers present in the first sheet (13) is 24 g/m² or less, or 22 g/m² or less, or 18 g/m² or less, and/or at least 5 g/m² or at least 7 g/m², or at least 10 g/m².

Suitable first sheets include, for example: a nonwoven sheet comprising a 17 or 22 gsm (g/m²) SMMMS or SMMS nonwoven layer attached to (but not laminated to) another 17 or 22 gsm SMMMS or SMMS nonwoven layer (wherein, for example, the meltblown level of each layer is 5.7 or 7.3 gsm respectively); a nonwoven sheet comprising 22 gsm SMMMS nonwoven layer, with, for example, 7.3 gsm meltblown fibers, attached to 17 gsm SMMMS or SMMS nonwoven layer, comprising for example 5.7 gsm meltblown fibers; a nonwoven sheet comprising a 17 gsm or 22 gsm SMS or SNS nonwoven layer, attached to another 17 gsm or 22 gsm SNS or SMS nonwoven layer.

The first sheet (13) may comprise a hydrophobic surface coating, such as known in the art, for example a wax, or a hydrophobic surface coating comprising one or more silicone polymers or fluorinated polymers. Suitable silicone polymers are, for example, selected from the group consisting of silicone MQ resins, polydimethysiloxanes, crosslinked silicones, silicone liquid elastomers, and combinations thereof. Typically, the molecular weight of such silicone polymers should be at least about 4,000 MW, or at least about 10,000 MW, or at least about 15,000 MW, or at least about 20,000 MW, or at least about 25,000 MW. Polydimethylsiloxanes are selected from the group consisting of vinyl-terminated polydimethsiloxanes, methyl hydrogen dimethylsiloxanes, hydroxyl-terminated polydimethysiloxanes, organo-modified polydimethylsiloxanes, and combinations thereof. Suitable fluorinated polymers are selected from the group consisting of telomers and polymers containing tetrafluoroethylene and/or perfluorinated alkyl chains. For instance, fluorinated surfactants, which are commercially available from Dupont under the tradename Zonyl®, are suitable for use herein. In particular, Zonyl® 321, 329, 8740, 9027, and 9360 are suited for use herein. Additionally, other Zonyl® materials include fluoroadditives like micro-powders may be useful herein. These include, but are not limited to Zonyl® MP1100, MP1200, MP1400, MP1500J, MP1600N, TE-3667N (which is a water dispersion). In one embodiment, the coating is free of aminosilicones.

These materials are deposited onto the composite sheet (10) in amounts of from at least about 0.01 gsm (gram of material/square meter of composite sheet), or from at least about 0.05 gsm, or from at least about 0.1 gsm.

First sheets or composite sheets (10) are considered urine-impermeable and feces impermeable, when they have a low surface energy and a uniform pore size distribution, and may have the low surface energy values, pore sizes and air permeability values described in co-pending application EP-A-1417945. For example, materials may be used that are substantially impermeable materials with an alcohol repellency of at least 5, or at least 6, or at least 7, or at least 8; and, for example, having a surface energy of between 20 and 35 mN/m; optionally having a contact angle with water of above 100°; and optionally having a mean pore size of less than 50 microns, or less than 30 microns, or less than 20 microns, but optionally at least 2 microns, or at least 5 microns. The first sheet (13) or composite sheet (10) may have an air permeability of at least 3 Darcy, or at least 10 Darcy, or at least 20 Darcy, or at least 30 Darcy.

Absorbent Articles

The absorbent article may be any absorbent article that is to be worn in contact or close proximity to the skin of a wearer of the article, and that may benefit from having elasticity. Absorbent articles herein include feminine hygiene articles, such as panty-liners and sanitary napkins or pads, adult incontinence products, such as briefs, pads or diapers, and baby diapers, toddler diapers with fasteners, and pull-on diapers (or pants).

The absorbent article may comprise the composite sheet (10) typically at the surface that faces the wearer in use, e.g. such that the composite may contact the skin of the user.

In one embodiment, the composite sheet (10) may form, or be part of, one or more of the cuffs of the absorbent article, such as the barrier cuff and/or leg cuff. Alternatively, or in addition, the composite sheet (10) may form, or be part of, the waistband of the absorbent article. The waistband(s), the barrier cuff(s) and/or leg cuff(s) may each be such that at least 40%, and up to 100%, of the surface area of a cuff is formed by the composite sheet (10).

The barrier cuff, comprising or being formed from the composite sheet (10) herein, may be in any shape or dimension known in the art. It may be that the barrier cuff is posited adjacent a longitudinal edge of the absorbent core, and extending longitudinally along at least 70% of the length of the article. There may be a pair of opposing barrier cuffs, each extending longitudinally and being positioned adjacent either side of the absorbent core. The cuff may have a free longitudinal edge that can be positioned out of the X-Y plane (longitudinal/transverse directions) of the article, i.e. in z-direction. The barrier cuffs of a pair may be mirror images of one another in the Y-axis of the article.

The absorbent article may alternatively, or in addition, comprise one or more leg cuffs, typically a pair of opposing leg cuffs, each being positioned adjacent one longitudinal side of the absorbent core, and extending longitudinally along the core, and each being positioned outwardly from a barrier cuff, if present. The leg cuffs may extend longitudinally along at least 70% of the length of the article. The leg cuff(s) may have a free longitudinal edge that can be positioned out of the X-Y plane (longitudinal/transverse directions) of the article, i.e. in z-direction. The leg cuffs of a pair may be mirror images of one another in the Y-axis of the article.

For clarity, if the absorbent article herein comprises a pair of leg cuffs or a pair of barrier cuffs, or a front waist band and a back waist band, then the article typically comprises a pair of composite sheets (10), as described herein, each having at least one elasticized region (12a or 12b).

The elasticized regions (12a, 12b) of the composite sheet (10) may comprise a covering sheet, or second sheet (14), material on the side of the elastic material (15) of the region, that is not facing (and partially adhered to) the first sheet, as described above.

Any portion of the composite sheet may be coated with a skin care composition or lotion or powder. Such a skin care composition or lotion may be present at least on the elasticized regions (12a, 12b), and possibly on the secondary elasticized regions and other regions. Examples of lotions include those described in U.S. Pat. No. 5,607,760; U.S. Pat. No. 5,609,587; U.S. Pat. No. 5,635,191; U.S. Pat. No. 5,643,588; WO 95/24173, provided the lotion is compatible with the elastic material (15), and does not destroy the elastic material (15) or reduce its elasticity.

Exemplary absorbent articles comprise at least a topsheet, facing the wearer in use, for example a nonwoven sheet, and/or an apertured sheet, including apertured formed films, as known in the art, and a backsheet, an absorbent core, having optionally a core coversheet facing the wearer in use.

The backsheet may be liquid impervious, as known in the art. In some embodiments, the liquid impervious backsheet comprises a thin plastic film such as a thermoplastic film having a thickness of about 0.01 mm to about 0.05 mm. Suitable backsheet materials comprise typically breathable material, which permit vapors to escape from the diaper while still preventing exudates from passing through the backsheet. Suitable backsheet films include those manufactured by Tredegar Industries Inc. of Terre Haute, Ind. and sold under the trade names X15306, X10962 and X10964.

The backsheet, or any portion thereof, may be elastically extendable in one or more directions. The backsheet may be attached or joined to a topsheet, the absorbent core, or any other element of the diaper by any attachment means known in the art. It may be that the longitudinal side edges of the topsheet and backsheet are directly attached to one another, but that the longitudinal edges of the topsheet and the core are not attached to one another.

The absorbent core may comprise any absorbent material which is generally compressible, conformable, non-irritating to the wearer's skin, and capable of absorbing and retaining urine, such as comminuted wood pulp, creped cellulose wadding; melt blown polymers, including coform; chemically stiffened, modified or cross-linked cellulosic fibers; tissue, including tissue wraps and tissue laminates; absorbent foams; absorbent sponges; super absorbent polymers; absorbent gelling materials; or any other known absorbent material or combinations of materials; the absorbent core may have an absorbent storage layer which comprises more than 80% by weight of the absorbent core content (e.g. excluding core wrap) of absorbent gelling material, and may be free of airfelt.

The absorbent article may also include a sub-layer disposed between the topsheet and the absorbent core, capable of accepting, and/or immobilizing bodily exudates, typically fecal material. Suitable materials for use as the sub-layer may include large cell open foams, macroporous compression resistant non woven highlofts, large size particulate forms of open and closed cell foams (macro and/or microporous), highloft non-wovens, polyolefin, polystyrene, polyurethane foams or particles, structures comprising a multiplicity of vertically oriented, possibly looped, strands of fibers, or apertured formed films, as described above with respect to the genital coversheet. (As used herein, the term “microporous” refers to materials that are capable of transporting fluids by capillary action, but having a mean pore size of more than 50 microns. The term “macroporous” refers to materials having pores too large to effect capillary transport of fluid, generally having pores greater than about 0.5 mm (mean) in diameter and more specifically, having pores greater than about 1.0 mm (mean) in diameter, but typically less than 10 mm, or even less than 6 mm (mean).

The diapers herein may comprise a waistband, which may be an elasticized waistband, comprising the composite sheet (10) herein. The diapers herein may have a fastening system, typically joined to the waistband, as known in the art. The fastening system may comprise fastening tabs and landing zones, wherein the fastening tabs are attached or joined to the back region of the diaper and the landing zones are part of the front region of the diaper.

The articles (e.g. diaper) when packed in their packaging material, may comprise two transverse folds, so that when unfolded for use by the user or care taker, the article (e.g. diaper) is in a U-shape and easier to apply.

Test Methods

Handle-O-Meter Bending Rigidity Test

This method serves to determine the bending rigidity (and thereby softness) of a nonwoven layer or nonwoven sheet, and reflects the flexibility and surface friction of the material. In this test, a nonwoven is deformed through a slot by use of a plunger, and the required force is measured. This method is based on the INDA Standard test IST 90.3-92.

A sample material of the nonwoven sheet or nonwoven layer of 1 inch long and 1 inch wide (25 mm×25 mm) is cut and conditioned at 65% humidity and 21° C. as set out in the INDA test. The sample is free form elastic material (15) or edges attached to other materials. In one embodiment, the average fiber direction of the nonwoven web or layer in contact with the skin in use can be determined and this would be the Y-direction (e.g. typically corresponding to the MD dimension of the absorbent article).

A handle-o-meter, available from Twingh-Albert Instruments Co., Philadelphia, USA, is calibrated as set out in the user instructions.

The slot width is 6.35 mm.

The sample is placed under the plunger and on the slot with the surface that in use contacts or faces the skin facing up. A first dimension is perpendicular to the slot and this is the direction tested, for which the bending rigidity is reported herein. In one embodiment, this is the average fiber direction of the skin-facing surface, e.g. the spunbond layer. The sample is centered over the slot and the test is run and the force is measured. This value is multiplied by 4 (e.g. normalised to a 4 inch×4 inch sample) and reported in grams herein as the bending rigidity.

Hydrostatic Head (Hydrohead)

The hydrostatic head (also referred to as hydrohead) is measured with a low surface tension liquid, i.e. a 49 mN/m liquid (solution).

This liquid is prepared as set out below.

This test is performed as set out in co-pending application WO2005/112854A, which conforms to the INDA/EDANA test WSP 80.6 (05). However, the water pressure (from below) is increased at a rate of 60 mbar/min.

A sample of 5 cm² is taken from the composite sheet or first sheet. The sample should be free from elastic material (15) or edges that are connected to other materials.

The test head used has a 2.5 cm diameter; the protective sleeve used has a 2.2 cm diameter.

The test is performed on this sample and the Hydrostatic head value is obtained.

49 mN/m (dynes/cm) Liquid Preparation:

A 10 litre canister with tap is cleaned thoroughly 3 times with 2 litres polyethylene and then 3 times with 2 litres distilled/deionized water.

Then, it is filled with 10 litres distilled/deionized water and stirred with a clean stirring bar for 2 h, after which the water is released via the tap.

A 5 litre glass is cleaned 6 times with water and then 6 times with distilled/deionized water.

Then, 30.00 g of Na Cholate and 5 litres of distilled/deionized water are placed in the cleaned 5 litres glass. (NaCholate should have a TLC purity of >99%, e.g. supplied by Calbiochem, catalog #229101). This is stirred with a clean stirring bar for about 5 min, until the Na Cholate is visibly dissolved.

The stirring bar is removed from the glass with a magnetic stick (without touching the solution) and then the Na cholate solution is poured into the 10 litres canister and more distilled/deionized water is added such that the concentration of the final solution is 3 g/l. This is further stirred with a stirring bar for 2 hours and then used.

This preparation of the solution and use thereof is at the temperature stated for the test for which it is used, or if no temperature is stated, it is kept at 20° C.

The surface tension of the solution is measured and this should be 49 mN/m (+/−2). The surface tension may be determined by method ASTM D1331-56 (“Standard test method for surface and interfacial tension of solution of surface active agents”) using a Kruss K12 tensiometer.

Strike Through Value Method

The low surface tension strike through value referred to herein may be obtained by the EDANA method WSP70.3 (05), except that a low surface tension liquid (see below) is used and a sample of 1 inch×1 inch (25 mm×25 mm) may be used. The sample should be free of elastic material (15) or of edges that are connected to other materials.

The low surface tension liquid is a liquid with a surface tension of 32 mN/m prepared as follows:

In a clean flask, 2.100 grams of Triton-X-100 is added to 500 ml distilled water (already in flask) and then 5000 ml distilled water is added. The solution is mixed for 30 minutes and then the surface tension is measured, which should be 32 mN/m.

(The surface tension may be determined by method ASTM D1331-56 (“Standard test method for surface and interfacial tension of solution of surface active agents”) using a Kruss K12 tensiometer.

Method to Measure Wrinkle Profile/Uniformity (Primos Method)

The wrinkle dimensions, e.g. height, and the wrinkle densities, and uniformity thereof, as described herein, can be measured as follows.

The composite sheet (10) with the elasticized region (12) is removed from the absorbent article such that the elongation potential, wrinkle height and wrinkle density are not changed. (If the PRIMOS equipment and method below can be used directly on the absorbent article with the composite sheet (10), then the composite sheet (10) does not need to be removed.)

It is left for 24 hours at 25° C. and 50% humidity, prior to the elongation/stretching step below, which will be performed under the same conditions.

One or more samples are marked in the partially stretched composite sheet (10), or cut therefrom, if necessary in order to do the PRIMOS measurement, as follows: the composite sheet (10) is gently and evenly stretched, horizontally and on a flat surface, to its fully stretched length and then released until it has 66.7% (2/3) of the fully stretched length. Then, one or more samples are marked in the composite sheet (10). The 66.7% stretched sample may be any length, but for example a sample may have a dimension of 7.5 cm in the direction of stretch (e.g. 7.5 cm of the length of the composite sheet (10) in 66.7% stretched state). A sample should have the full width of the elasticized region (12), and if possible, the full width of the composite sheet (10).

Measurement of lengths of the sample can be done with for example a micrometer screw.

The partially stretched sample, having 66.7% of its fully stretched length (for example a sample of 7.5 cm) is then examined by use of PRIMOS equipment and its data acquisition software, following the manufacturer's instruction manual, using a 13×18 mm lens.

The PRIMOS equipment and software will measure all peak heights, widths, etc., and the herein described values can be calculated therefrom. The height is the distance between the highest and lowest point of a peak. It should be noted that “shoulder peaks” are not regarded peaks of the wrinkles herein, as is a known approach in the art. Namely, if two adjacent peaks, A and B, are joined by a valley and either a) the distance from the highest point of peak A to the lowest point of the valley is less than 30% of the height of peak A or b) the distance from the highest point of peak B to the lowest point of the valley is less than 30% of the height of peak B, then the peak B is considered a shoulder peak and not an individual peak, and thus peak A and B are considered a single peak A (e.g. with a single width, single height etc.). Thus, if either a) or b) above applies, A and B are taken as one single peak.

Residual Strain

The residual strain of an absorbent article or of a composite sheet (10), obtainable by the process herein can be calculated as follows.

The elastic material (15) is conditioned as above. The contracted, relaxed length of the elastic material (15) or elasticized region (12) as used in the process to form the composite sheet (10) herein is determined. This is l₀.

Then, the length of the elastic material (15) or elasticized region (12) in the contracted absorbent article or composite sheet (10), conditioned as set out in the method above and on a flat surface, is measured. This is lₓ.

Then, the residual strain Sᵣ=[(lₓ−l₀)/lₓ] can be calculated.

For example, if 10 cm elastic material (15) is attached to a first sheet (13) over a length of 40 cm, and the resulting composite sheet (10) has a contracted length of 15 cm, then the residual strain is (15−10)/15=33.3%.

In a finished article, the residual strain can be calculated if the elastic material (15) can be removed from the article and then, the contracted length thereof can be calculated as above. (This of course after having calculated lₓ)

Peel Force Method

This method serves to determine the strength of the bond in the composite sheet (10) of the elastic material (15) and the first sheet (13 and second sheet (14); the peel force is the force required to undo the bond of (or delaminate) the elastic and the sheets.

The measurement may be done with for example a Zwick 2.5 KN tensile tester with a load cell of 50N. The test path is 100 mm. The speed is set to be 100 mm/min. The clamps are for example 25 mm×40 mm. The target gage length can suitably be set, e.g. 25 mm. (N.B.: In general, the load cell should be chosen in a way that the expected measurement values are in the calibrated range of the capacity of the load cell (e.g. 0.2-100% of capacity, which is for a 50 N load cell from 0.1 N-50 N)).

A sample is cut from the composite sheet (10) such that the whole width of the elasticized region (12) (and of the elastic material (15)) is comprised in the sample and such that at one longitudinal side (in direction of stretch) an area is present that is formed by the first and second sheet material, but not by the elastic material.

A JDC precision Sample cutter by Thwings-Albert Instrument Company, USA, may be used

A suitable sample may be 1 inch (25.4) long (in direction of stretch, Y-direction) and having the width of the elastic material (15) plus along one longitudinal side of the sample some of the neighboring first sheet (13) and second sheet that is not part of the elasticized region (12), e.g. attached to the elastic material (15), e.g. 40 mm.

The sample is conditioned for 16 hours at 50% relative humidity and 20° C.

Then the area formed by the first sheet and second sheet is carefully peeled open up to the elastic material; the peeled open area of the first sheet and second sheet are attached between the clamps of the test equipment, so that there is no slack in the sample. The test can then be run and the force to peel the elastic material and the first sheet and/or second sheet is recorded and reported as peel force value used herein. If the composite sheet allows more samples to be taken, then this test can be repeated for more samples, and the average peel force can be obtained, and reported as peel force values.

The dimensions and values disclosed herein are not to be understood as being strictly limited to the exact numerical values recited. Instead, unless otherwise specified and not with respect to the test methods above, each such dimension is intended to mean both the recited value and a functionally equivalent range surrounding that value. For example, a dimension disclosed as “40 mm” is intended to mean “about 40 mm”.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A process for making a composite sheet useful for an absorbent article, the composite sheet comprising a wrinkled, patterned elasticized region that comprises a patterned first sheet and an elastic material, comprising the steps of:
a) obtaining a first sheet with a Y-direction and X-direction;
b) obtaining an elastic material that is at least partially stretched, having at least an average longitudinal direction of stretch Y substantially along the Y direction of the sheet;
c) i) submitting the first sheet or part thereof to a patterning, pressure-applying step to obtain a patterned first sheet comprising troughs, and then positioning the at least partially stretched elastic material adjacent the patterned first sheet to obtain a combined material; or
ii) positioning the at least partially stretched elastic material adjacent the first sheet to obtain a combined material and simultaneously or subsequently submitting the combined material, or part thereof, to a patterning, pressure-applying step to obtain a patterned combined material, comprising a patterned first sheet comprising troughs;
d) simultaneously or subsequent to step c) attach the troughs, or part thereof, of the patterned first sheet to the elastic material, to thus obtain a stretched composite sheet comprising a patterned elasticized region; and
e) relaxing the composite sheet of step d) to obtain a composite sheet comprising a wrinkled, patterned elasticized region, comprising wrinkles with peaks and valleys, whereby the valleys are formed by both the elastic material and the troughs of the first sheet.

2. A process as in claim 1, comprising the step c ii) wherein the first sheet and elastic material are positioned adjacent one another prior to or simultaneously with the patterning, pressurizing step and whereby the step c ii) and d) are performed simultaneously.

3. A process as in claim 1, wherein in step c) a first surface of the first sheet or part thereof, that is not facing the elastic material, is pressurized, and optionally contacted, by a first surface of a first tool, having a multitude of raised portions, whereby the raised portions form the troughs in the first sheet or part thereof; and wherein a second surface of the first sheet, facing the elastic material is pressurized, and optionally indirectly contacted by a second, non-mating surface of a second tool.

4. A process as in claim 3, wherein the first tool has a transverse axis and wherein the raised portions are in the form of: a) a multitude of ridges, each ridge being positioned under angle of between 0° to 45° with a line parallel to the transverse axis, and/or b) a multitude of teeth and/or studs, positioned along the X and Y direction of the tool's surface, in first rows positioned under angle of between 0° to 45° with a line parallel to the transverse axis and second rows positioned perpendicular to the first rows.

5. A process as in claim 3, wherein the distance between the raised portions and the second tool's surface is 0.01 mm to 0.25 mm.

6. A process as in claim 3, wherein the raised portions include a first flat surface in x-y plane that contacts the sheet material first, the first surface having a Y-dimension substantially perpendicular to the X-direction of the tool, of from 0.05 mm to 0.5 mm, and wherein when the raised portions are studs or teeth, having an X-dimension of from 0.05 mm to 2.0 mm.

7. An absorbent article comprising a composite sheet comprising a wrinkled, patterned elasticized region, having a longitudinal direction Y and a transverse direction X, and comprising a first sheet and an elastic material, the elastic material extending at least partially in substantially the longitudinal direction Y and providing elasticity in substantially the longitudinal direction Y, wherein the first sheet, or part thereof, comprises in longitudinal direction, and optionally in transverse direction, a pattern of a multitude of troughs and a multitude of crests, the troughs of the first sheet having a higher density than the crests of the first sheet; and wherein the elastic material is attached to the troughs of the first sheet, and wherein the wrinkled elasticized region comprises wrinkles that have peaks and valleys, the troughs corresponding with the valleys.

8. An absorbent article as in claim 7, wherein the elasticized region of the composite sheet comprises two or more elastic strings or strands, extending in substantially the Y-direction of the composite sheet, and the composite sheet has a multitude of troughs along the Y-direction and multitude of troughs along the X-direction, wherein one or more troughs, or part thereof, in the X-direction is present between the elastic strings or strands.

9. An absorbent article as in claim 7, wherein the first sheet in the elasticized region comprises, at an elongation of 66.67%, a uniform wrinkle pattern, having wrinkles of an average wrinkle height H_{w} of from 150 microns to 600 microns and having a wrinkle height standard deviation (STD) of less than 100 microns, and optionally, the RSD is less than 20%.

10. An absorbent article as in claim 7, whereby the first sheet in the elasticized region comprises, at an elongation of 66.67%, a uniform wrinkle pattern, having wrinkles with peaks and valleys, wherein the average distance between the highest points of neighboring peaks of the wrinkles is 800 to 1300 microns, and wherein the standard deviation is less than 200 microns and wherein the RSD is less than 20%.

11. An absorbent article claim 7, wherein the composite sheet is a composite barrier sheet, having a hydrostatic head of at least 15 mbar and/or the first sheet is a barrier sheet having a hydrostatic head of at least 15 mbar.

12. An absorbent article as in claim 7, wherein the first sheet is a nonwoven sheet or nonwoven layer having at least two spunbond webs and at least one meltblown web, the sheet or layer having a basis weight of at least 17 g/m², and the sheet or layer comprising at least 3 g/m² meltblown fibers.

13. An absorbent article comprising a composite sheet that comprises a patterned and wrinkled elasticized region, the elasticized region containing an elastic material and a patterned and wrinkled first sheet, having troughs that are attached or partially attached to the elastic material, and wherein the elasticized region has a residual strain of less than 30%, further wherein the composite sheet comprises a wrinkled second sheet, that may optionally be patterned, and wherein the elastic material is positioned between the first sheet and the second sheet, and wherein the elasticized region has a peel force of at least 2.0 N.

14. A patterning tool, having an X-direction and Y-direction, the tool being a roll with an X-direction-axis, the tool having raised portions in rows substantially along the X-direction and in columns substantially along the Y-direction, wherein the X-direction height of a raised portion is less than the Y-direction height of the same raised portion and the ratio of the Y-direction height to the X-direction height is at least 3:2, and the X-direction height is from 0.1 mm to 1.0 mm and the Y-direction height is from 0.4 mm to 3 mm.

15. A tool as in claim 22, which comprises a cooling unit, wherein the tool is cooled by the cooling unit.
